(19) United States
(12) Patent Application Publication
(10) Pub. No.: US 2023/0340225 A1
(43) Pub. Date: Oct. 26, 2023

(54) METHOD FOR MANUFACTURING FLEXIBLE SENSOR

(71) Applicant: QILU UNIVERSITY OF TECHNOLOGY, Jinan (CN)

(72) Inventors: Li WANG, Jinan (CN); Jun CHEN, Jinan (CN); Weiguang SU, Jinan (CN); Chonghai XU, Jinan (CN); Wenhong ZHANG, Jinan (CN); Junlei HAN, Jinan (CN); Xingyuan XU, Jinan (CN)

(73) Assignee: QILU UNIVERSITY OF TECHNOLOGY, Jinan (CN)

(21) Appl. No.: 18/217,653

(22) Filed: Jul. 3, 2023

Related U.S. Application Data
(63) Continuation of application No. PCT/CN2022/132016, filed on Nov. 15, 2022.

(30) Foreign Application Priority Data
Nov. 17, 2021 (CN) 2021113632298

(51) Int. Cl. C08K 3/04; C08J 3/07; C08J 3/28

(52) U.S. Cl. CPC C08K 3/042; C08J 3/07; C08J 3/28; C08J 2383/04; C08J 2333/12

(57) ABSTRACT

A method for manufacturing a flexible sensor, including: dispersing graphene in a polymeric material; adding a carbon nanotube into the polymeric material; applying an alternating electric field to the polymeric material added with the carbon nanotube to obtain a composite material; attaching a polymeric material film to the obtained composite material; pre-embedding carbon fibers; and heating and curing to obtain a sensor. A CNT bridging effect and an electric field induced arrangement are introduced at an appropriate ratio of polymeric materials such as graphene-PDMS or PMMA to improve a dry-blended method. The flexible sensor manufactured by the improved dry-blended method improves electrical conductivity, a piezoresistive property and a mechanical property of CNT-graphene-PDMS or PMMA and other polymeric materials.

### CROSS-REFERENCE TO RELATED APPLICATIONS

The present disclosure claims priority to the Chinese Patent Application No. 202111363229.8, entitled “METHOD FOR MANUFACTURING FLEXIBLE SENSOR”, filed to the China Patent Office on Nov. 17, 2021, the entire content of which is incorporated herein by reference.

### TECHNICAL FIELD

The present disclosure belongs to the technical field of sensor manufacturing and particularly relates to a method for manufacturing a flexible sensor.

### BACKGROUND

Flexible sensors based on graphene or carbon nanotubes (CNT) have been widely used for resistance strain measurement in the field of human health monitoring because of their unique characteristics in electrical conductivity and piezoresistivity. A key process in preparation of conductive flexible sensors is to uniformly disperse solid powdery conductive tillers into a viscous polymer liquid at a micro scale to form a conductive matrix network in a flexible substrate (such as graphene-polydimethylsiloxane (PDMS) or polymethyl methacrylate (PMMA) and other polymeric materials).

An organic solvent-based method is the most common and conventional method for preparing a conductive flexible sensor, and it involves using various organic solvents (ethylene glycol, toluene, tetrahydrofuran, and n-hexane) as diluents to dilute a viscous prepolymer liquid of the polymeric materials such as PDMS or PMMA to uniformly, distribute the fillers. However, the above organic solvents are toxic and harmful to experimenters, the environment and biological samples, for example, in a curing process, the polymeric materials such as graphene-PDMS or PMMA are heated in vacuum to volatilize the organic solvents, which should be operated under strict protective conditions to avoid causing harm to operators. Therefore, there is an urgent need to develop a new method for preparing the polymeric materials such as graphene-PDMS or PMMA without using toxic solvents. A non-toxic dry-blended method is one of the most promising methods, which directly mixes graphene with the polymeric materials such as PDMS or PMMA without diluting organic solvents.

The inventors of the present disclosure have found that there are the following problems when using a non-toxic dry-blended method to manufacture the flexible sensors: compared with methods based on solvents, a current dry-blended method results in significant reduction in electrical and piezoresistive properties of a polymeric material such as graphene-PDMS or PMMA.

### SUMMARY

In order to solve the above problems, the present disclosure proposes a method for manufacturing a flexible sensor. According to the present disclosure, a CNT bridging effect and an electric field induced arrangement are introduced at an appropriate ratio of polymeric materials such as graphene-PDMS or PMMA to improve a dry-blended method. The flexible sensor manufactured by the improved dry-blended method improves electrical conductivity, a piezoresistive property and a mechanical property of the polymeric material such as CNT-graphene-PDMS or PMMA.

In order to realize the above objectives, the present disclosure is implemented through the following technical solutions.

In a first aspect, the present disclosure provides a method for manufacturing a flexible sensor, including:
dispersing graphene in a polymeric material;
adding a carbon nanotube into the polymeric material;
applying an alternating electric field to the polymeric material added with the carbon nanotube to obtain a composite material;
attaching a polymeric material film to the obtained composite material;
pre-embedding carbon fibers; and
heating and curing to obtain a sensor.

Further, the graphene is dispersed in the polymeric material in a mode of mechanical stirring and high-power ultrasound by using a dry-blended method.

Further, a mechanical stirring duration is 0.5 hour to 24 hours.

Further, the polymeric material is polydimethylsiloxane or polymethyl methacrylate.

Further, a mass ratio of the graphene is 2 wt % to 30 wt %,

Further, a mass ratio of the carbon nanotube is 0.1 wt % to 10 wt %.

Further, the alternating electric field is set as a sinusoidal complex-frequency alternating electric field with an intensity of 1⁰⁴ to 10⁶ V/m and a frequency of 100 Hz to 10 KHz.

Further, a curing agent is added into the mixed composite material before use, and a mass ratio of the composite material to the curing agent ranges from 5 wt % to 20 wt %.

Further, a process of attaching the polymeric material film to the obtained composite material is that the composite material is put into a mold, and the polymeric material film is attached to a top of the mold.

Further, a process of heating and curing is that the polymeric material film is compacted, and the carbon fibers are embedded at two ends for curing at 70° C. for 5 hours.

In a second aspect, a flexible sensor is prepared by the method described in first aspect. Compared with the prior art, the present disclosure has the beneficial effects: according to the present disclosure, the CNT bridging effect and the electric field induced arrangement are introduced at the appropriate ratio of the polymeric material such as graphene-PDMS or PMMA to improve the dry-blended method. The flexible sensor manufactured by the improved dry-blended method improves electrical conductivity, a piezoresistive property and a mechanical property of the polymeric material such as CNT-graphene-PDMS or PMMA.

### BRIEF DESCRIPTION OF THE DRAWINGS

Accompanying drawings, which constitute a part of the present disclosure, of the specification are intended to provide a further understanding of the present disclosure. The illustrative embodiments and descriptions of the present disclosure are used to explain the present disclosure, and do not constitute an improper limitation to the present disclosure.

FIG. 1 is a schematic diagram of electrical conductivity (0.1 wt %, 0.25 wt %, 0.5 wt %, 1 wt %, 2 wt % and 3 wt % CNT added into a polymeric material such as 10.5 wt % graphene-PDMS or PMMA) of carbon nanotube bridged polymeric material such as graphene-PDMS or PMMA, and the polymeric material such as graphene PDMS or PMMA (from 10,6 wt % to 13.5 wt %) in embodiment I of the present disclosure.

FIG. 2 is a schematic diagram showing distribution and comparison of an electrical conductivity ratio (ECR) of carbon nanotube bridged polymeric material such as graphene-PDMS or PMMA of adjacent weight ratios or adjacent polymeric materials such as graphene-PDMS or PMMA in embodiment 1 of the present disclosure.

FIG. 3 is a schematic diagram of an example of a piezoresistance test of 1 wt % CNT-bridged polymeric material such as graphene-PDMS or PMMA and a polymeric material such as 11.5 wt % graphene-PDMS or PMMA in embodiment 1 of the present disclosure.

FIG. 4 is a schematic diagram of an example of a piezoresistance test of 1 wt % CNT-bridged polymeric material such as graphene-PDMS or PMMA and a polymeric material such as 11.5 wt % graphene-PDMS or PMMA in embodiment 1 of the present disclosure.

FIG. 5 is a schematic diagram of a piezoresistive strain range ratio of carbon nanotube bridged polymeric material such as graphene-PDMS or PMMA and a polymeric material such as graphene-PDMS or PMMA in embodiment 1 of the present disclosure.

FIG. 6 is a schematic diagram of a strain coefficient (sensitivity index) ratio of carbon nanotube bridged polymeric material such as graphene-PDMS or PMMA to a polymeric material such as graphene-PDMS or PMMA in embodiment 1 of the present disclosure.

FIG. 7 is a schematic diagram of property comparison (electrical conductivity, a piezoresistive property and a mechanical property) of CNT-bridged polymeric material such as graphene-PDMS or PMMA with or without an electric field in embodiment 1 of the present disclosure.

FIG. 8 is a schematic diagram of an example showing a peizoresistance test of aligned 1 wt % CNT-bridged polymeric material such as graphene-PDMS or PMMA induced by an electric field in embodiment 1 of the present disclosure.

FIG. 9 is a schematic diagram of scanning electron microscope (SEM) imaging showing that CNT bridges adjacent polymeric material clusters such as graphene-PDMS or PMMA in embodiment I of the present disclosure.

FIG. 10 is a schematic diagram of SEM imaging showing that aligned and parallel carbon nanotubes bridge adjacent polymeric material clusters such as graphene-PDMS or PMMA in an electric field direction in embodiment 1 of the present disclosure.

FIG. 11 is a schematic diagram of a dynamic rearrangement process (magnified by 100 times): 0 minute of CNT and graphene using a microscale electric field under an optical microscope in embodiment I of the present disclosure.

FIG. 12 is a schematic diagram of a dynamic rearrangement process (magnified by 100 times): 5 minute of CNT and graphene using a microscale electric field under an optical microscope in embodiment 1 of the present disclosure.

FIG. 13 is a schematic diagram of a dynamic rearrangement process (magnified by 100 times): 10 minute of CNT and graphene using a microscale electric field under an optical microscope in embodiment 1 of the present disclosure.

FIG. 14 is a schematic diagram of a dynamic rearrangement process (magnified by 100 times): 20 minutes after electric field treatment of CNT and graphene using a microscale electric field under an optical microscope in embodiment 1 of the present disclosure.

FIG. 15 is a schematic diagram of a stained nucleus of 7-day cardiomyocytes in embodiment 1 of the present disclosure.

FIG. 16 is a schematic diagram of a stained nucleus of 7-day cardiomyocytes in embodiment 1 of the present disclosure.

FIG. 17 is a schematic diagram of comparison of a density of cells cultured on CNT-bridged polymeric material such as graphene PDMS or PMMA and the polymeric material such as graphene PDMS or PMMA by the solvent-based method on the first day and the seventh day in embodiment 1 of the present disclosure.

FIG. 18 is a schematic diagram of carbon nanotube bridged polymeric material such as graphene PDMS or PMMA and a solvent-based polymeric material such as graphene PDMS or PMMA in a stained a-actinin confocal image of cardiomyocytes after 7 days of culture in embodiment 1 of the present disclosure.

FIG. 19 is a schematic diagram of carbon nanotube bridged polymeric material such as graphene PDMS or PMMA and a solvent-based polymeric material such as graphene PDMS or PMMA in a stained a-actinin confocal image of cardiomyocytes after 7 days of culture in embodiment 1 of the present disclosure.

FIG. 20 is a schematic diagram of an average sarcomere length, measured on the seventh day, of cardiomyocytes cultured on CNT-bridged polymeric material such as graphene PDMS or PMMA and solvent-based polymeric material such as graphene PDMS or PMMA (1.66±0.11 μm and 1.86±0.10 μm) in embodiment 1 of the present disclosure.

FIG. 21 is a schematic diagram of cardiomyocytes cultured on CNT-bridged polymeric material such as graphene PDMS or PMMA and a solvent-based polymeric material such as graphene PDMS or PMMA from the first day to the seventh day in embodiment I of the present disclosure.

FIG. 22 is a schematic diagram when CNT bridges a polymeric material such as graphene PDMS or PMMA and a solvent FT-IR spectrum is based on the polymeric material such as graphene PDMS or PMMA in embodiment 1 of the present disclosure.

FIG. 23 is a schematic diagram of membrane deflection caused by diastolic and systolic phases of cardiac contraction at a cellular level in embodiment 1 of the present disclosure.

FIG. 24 is a schematic example diagram of measurement of ΔR/R0 caused by cardiomyocytes contraction in a culture process, and a CNT electrical resistance signal on the first, third and fifth days in embodiment 1 of the present disclosure.

FIG. 25 is a schematic diagram (organ level) showing measurement of cardiac contraction in a mouse heart and a schematic diagram of connecting a flexible device to a heart surface of an anesthetized rat in embodiment 1 of the present disclosure.

FIG. 26 is a schematic diagram of measurement of ΔR/R0 generated by cardiac contraction until an anesthetized rat stops beating in embodiment 1 of the present disclosure.

### DETAILED DESCRIPTION

The present disclosure is further illustrated below in conjunction with the accompanying drawings and embodiments.

The following detailed descriptions are illustrative and are intended to provide further explanation of this application. Unless otherwise specified, all technical and scientific terms used herein have the same meanings as commonly understood by a person of ordinarily skilled in the art to which this application belongs.

### Embodiment 1

This embodiment provides a method for manufacturing a flexible sensor, including:
graphene is dispersed in a polymeric material;
adding a carbon nanotube into the polymeric material;
applying an alternating electric field to the polymeric material added with the carbon nanotube to obtain a composite material;
a polymeric material film is attached to the obtained composite material;
carbon fibers are pre-embedded; and
heating and curing are performed to obtain a sensor.

In this embodiment, specific steps are: the graphene is dispersed in the polymeric material such as PDMS or PMMA in a mode of mechanical stirring and high-power (greater than 300 W) ultrasound by using a dry-blended method. Preferably, a stirring duration is 0.5 hour to 24 hours, a mass ratio of the graphene is 2 wt % to 30 wt %, and a mass ratio of the carbon nanotube as a bridge is 0.1 wt % to 10 wt %. In a process for preparing polymeric composite materials such as graphene-CNT-PDMS or PMMA, a sinusoidal complex-frequency alternating electric field with an electric field intensity of 10⁴ to 10⁶ V/m and a frequency of 100 Hz to 10 KHz is applied. For consistency and reference of subsequent testing, standard samples with the same size are cast from the above composite materials with different graphene mass fractions respectively. A curing agent is added into the mixed composite material before use, and a mass ratio of the composite material to the curing agent ranges from 5 wt % to 2.0 wt %. For the consistency and reference of subsequent testing, standard samples with the same size are cast from the above polymeric composite materials such as graphene-PDMS or PMMA with different CNT mass ratios respectively, Then, the composite material is put into a mold of 0.02 mm×3 mm×30 mm prepared by a photoetching technology, and a polymeric material film such as PDMS or PMMA of 0.005 mm to 0.1 mm prepared by a spin coater is attached to a top of the mold. It is carefully, compacted with pointed tweezers, the carbon fibers are embedded at two ends with an embedded depth of 0.5 cm, and then it is put into an oven and cured at 70° C. for 5 hours.

A dry-blended preparation process is improved through two stages, including 1, a small quantity of carbon nanotubes are doped into a polymeric material such as graphene-PDMS or PMMA at an appropriate ratio of the polymeric material such as graphene-PDMS or PMMA (CNT-bridged polymeric material such as graphene-PDMS or PMMA), which will fill a gap between adjacent graphene of the polymeric materials such as graphene-PDMS or PMMA; and 2, the carbon nanotube bridged polymeric material such as graphene-PDMS or PMMA is enhanced by applying a high-frequency alternating electric field in a sample curing process to optimize a material ratio.

In this embodiment, key characteristics, such as electrical conductivity, a strain coefficient (piezoresistive sensitivity index), a piezoresistive range and a mechanical property, of the manufactured sensor of the carbon nanotube bridged polymeric material such as graphene-PDMS or PMMA are compared with those of a sensor manufactured by a method based on toxic organic solvents to verify the advantages of the method. In this embodiment, biocompatibility of the obtained sensor is also verified by culturing cardiomyocytes of a neonatal rat. Another flexible sensor manufactured by the proposed method has been shown to be capable of recording physiological activities, namely capturing a wide range of mechanical strain and bio-electrophysiological signals, The details are as follows:
the polymeric material such as graphene-PDMS or PMMA with different graphene weight ratios from 9 wt % to 16 wt % is prepared by the dry-blended method. The representation of electrical conductivity, and piezoresistive and mechanical properties proves that properties of the thy-blended method are worse than those of a solvent-based method, see Table 1. For example, the polymeric materials such as graphene-PDMS or PMMA prepared by the dry-blended method account for only 4.6% of the piezoresistive strain range compared to that prepared by tetrahydrofuran, and a half lethal dose of tetrahydrofuran to the rat is 1.6 g/kg.

TABLE 1
Property comparison of a polymeric material such as graphene-PDMS or PMMA prepared by a dry-blended
method with a polymeric material such as graphene-PDMS or PMMA prepared by a solvent-based method

| | | Weight | | Maximum | | Young's | Tensile | Elongation | |
|---|---|---|---|---|---|---|---|---|---|
| Blend | Organic | ratio | Electrical | strain | Gauge | modulus | strength | at break | |
| method | solventused | (wt %) | conductivity | range (%) | factor | (MPa) | (MPa) | (100%) | References |
| | | 9 | 3.30 × 10⁻⁷ | — | — | 2.53 | 2.15 | 0.92 | This work |
| | | 9.5 | 3.99 × 10⁻⁷ | — | — | 2.69 | 3.03 | 1.25 | This work |
| | | 10 | 1.88 × 10⁻⁶ | — | — | 3.11 | 1.64 | 0.57 | This work |
| | | 10.5 | 4.3 × 10⁻⁵ | — | — | 3.69 | 2.28 | 0.69 | This work |
| Dry- | — | 11 | 8.31 × 10⁻⁴ | — | — | 3.97 | 2.53 | 0.69 | This work |
| blended | — | 12 | 1.12 × 10⁻³ | 0-1.2 | 825.44 | 4.34 | 5.94 | 0.49 | This work |
| method | — | 13 | 1.55 × 10⁻³ | 0-1.5 | 533.69 | 5.20 | 1.87 | 0.4577278 | This work |
| | — | 14 | 1.58 × 10⁻³ | 0-1.5 | 504.74 | 5.89 | 2.31 | 0.4484111 | This work |
| | — | 15 | 3.96 × 10⁻³ | 0-2.3 | 364.41 | 7.42 | 2.33 | 0.3324861 | This work |
| | — | 16 | 4.12 × 10⁻³ | 0-2.5 | 323.09 | 9.06 | 1.65 | 23.71 | This work |
| Solvent- | N-hexane | 0.5 | 100 | 0-50 | 81 | — | — | 6 | 10 |
| basedmethod | N-hexane | 1 | — | — | — | 0.65 | 3.82 | 912 | 11 |
| | Chloroform | 1 | 1.6 ± 0.3 | 0-60 | 6.5 | — | — | — | 12 |
| | Formaldehyde | 10 | 0.8 | 0-20 | 20.5 | 1-13 | — | >30 | 13 |
| | Formaldehyde | 1.3 | 28 | 0-30 | 1 | — | — | — | 14 |
| | THF | 12 | 8.1 | 0-50 | — | 100 | — | 63 | 15 |
| | Toluene | 0.8 | 0.12 | 0-50 | 2 | 0.3 | — | 50 | 16 |
| | hydrazinehydrate | 0.33 | 1.2 × 10⁻⁴ | 0-10 | 4.3 | 3500 | — | — | 17 |

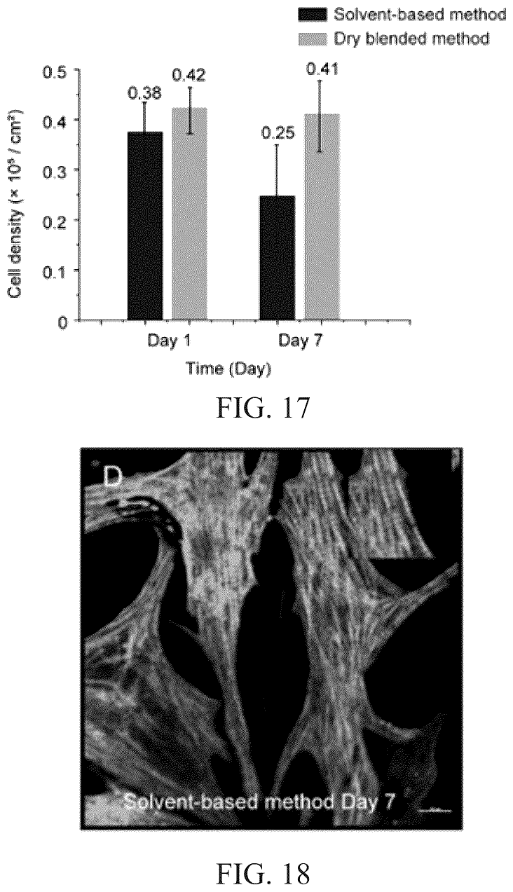

In order to improve the properties of the polymeric material such as graphene-PDMS or PMMA prepared by the dry-blended method, the carbon nanotubes (0.1 wt %, 0.25 wt %, 0.5 wt %, 1 wt %, 2 wt % and 3 wt %) are added and dispersed into the polymeric material such as graphene-PDMS or PMMA. The SEM imaging in FIG. 17 proves that the dispersed carbon nanotubes may build bridges between adjacent graphene clusters, so as to enhance a conductive network. The electrical conductivity, piezoresistive property and mechanical property (including Young's modulus, tensile strength and elongation at break) of the polymeric material samples such as carbon nanotube-graphene-PDMS or PMMA are measured. A polymeric material such as graphene-PDMS or PMMA with the same weight ratio (from 10.6 wt % to 13.5 wt %) is selected as a control group and measured.

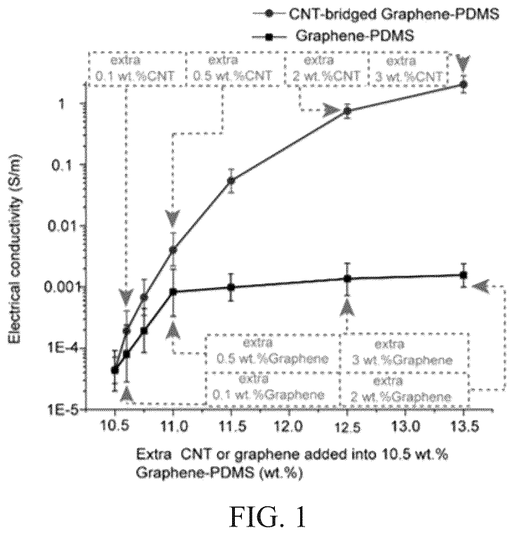

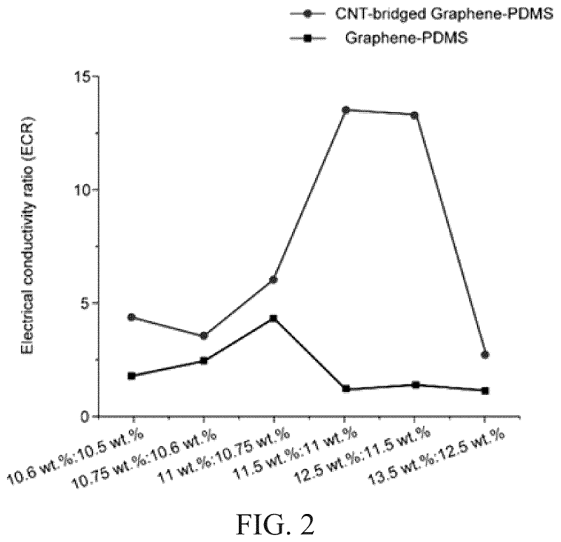

With the addition amount of CNT increasing from 0.1 wt % to 3 wt, the electrical conductivity of the CNT-bridged polymeric material such as graphene-PDMS or PMMA is 2.42 times (1.91×10⁻⁴±2.21×1.0⁻⁵ S/m vs 7.87×10⁻⁵±5.01×10⁻⁵ S/m) to 1296.62 times (2.02±0.57 S/m vs 1.56×10⁻³±0.61×10⁻³ S/m), which is greater than that of the polymeric material such as graphene-PDMS or PMMA. As shown in FIG. 1, the significant improvement in electrical conductivity is due to a one-dimensional tubular structure of CNT that may be bridged and connected to adjacent graphene of the polymeric material such as graphene-PDMS or PMMA (refer to SEM imaging in FIG. 17).

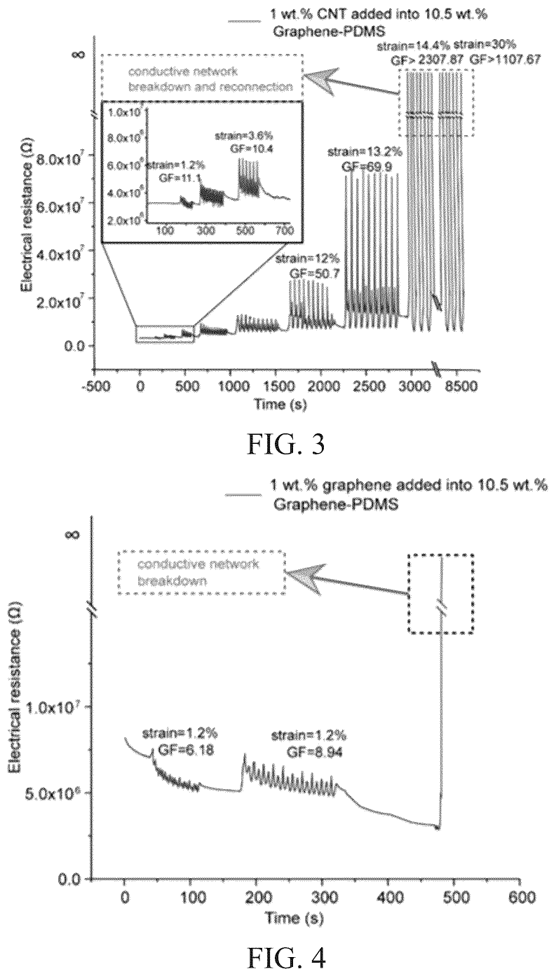

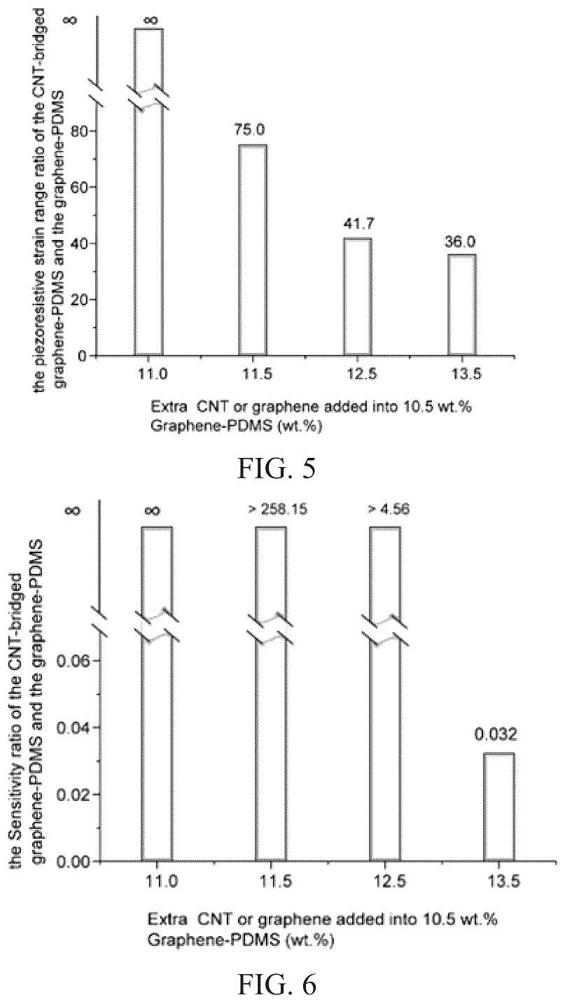

FIG. 3 shows a piezoresistive response curve for 0.1 wt %. The piezoresistive strain range and calculated strain coefficient are shown in Table 2. The 0.1 wt % and 0.25 wt % CNT-bridged polymeric material such as graphene-PDMS or PMMA have no significant piezoresistive response because no conductive network is formed. 0.5 wt %, 1 wt % and 2 wt % CNT-bridged polymeric material such as graphene-PDMS or PMMA generate regular electrical resistance increase within a strain range (for example, for the 1 wt % CNT-bridged polymeric material such as graphene-PDMS or PMMA, the strain range is from 0 to 13.2%, FIG. 3) Then, when strain further increases, the electrical resistance reaches a maximum electrical resistance range (1 GΩ) of a detection instrument. After the strain is released, the electrical resistance may be restored to an initial value (for example, 1 wt % CNT-bridged polymeric material such as graphene-PDMS or PMMA with the strain range from 14.4% to 30%, FIG. 3) In the comparison of a CNT bridging effect of the piezoresistive characteristic, FIG. 5 and FIG. 6 show that the piezoresistive strain range of the CNT-bridged polymeric material such as graphene-PDMS or PMMA is increased by at least 36 times compared to the corresponding control group (polymeric material such as 11 wt % to 13 wt % graphene-PDMS or PMMA). As shown in the figure, a maximum strain coefficient of the CNT-bridged polymeric material such as graphene-PDMS or PMMA is much higher than that of the corresponding control group (for example, 1 wt % CNT-bridged graphene-PDMS or PMMA and other polymeric material >2307.87 compared to 521 of a polymeric material such as 11.5 wt % graphene-PDMS or PMMA), in FIG. 6. Therefore, 1 wt % CNT is added into 10.5 wt % carbon nanotubes, which may significantly improve the piezoresistive strain range of the polymeric material such as graphene-PDMS or PMMA, and keep an infinite strain coefficient at the same time.

TABLE 2
Piezoresistive characteristics of carbon nanotube bridged
polymeric material such as graphene-PDMS or PMMA

| CNT-bridged graphene-PDMS or | Piezoresistive | Maximum |
|---|---|---|
| PMMA and otherpolymeric materials | strain range (%) | gauge factor |
| 0.1 wt % CNT added into 10.5 wt % | — | — |
| graphene-PDMS or PMMA and other | | |
| polymeric materials | | |
| 0.25 wt % CNT added into 10.5 wt % | — | — |
| graphene-PDMS or PMMA and other | | |
| polymeric materials | | |
| 0.5 wt % CNT added into 10.5 wt % | 0-4.8 | ∞ |
| graphene-PDMS or PMMA and other | | |
| polymeric materials | | |
| 1 wt % CNT added into 10.5 wt % | 0-30 | ∞ |
| graphene-PDMS or PMMA and other | | |
| polymeric materials | | |
| 2 wt % CNT added into polymeric | 0-48 | ∞ |
| materials such as 10.5 wt % | | |
| graphene-PDMS or PMMA | | |
| 3 wt % CNT added into polymeric | 0-54 | 16.8 |
| materials such as 10.5 wt % | | |
| graphene-PDMS or PMMA | | |

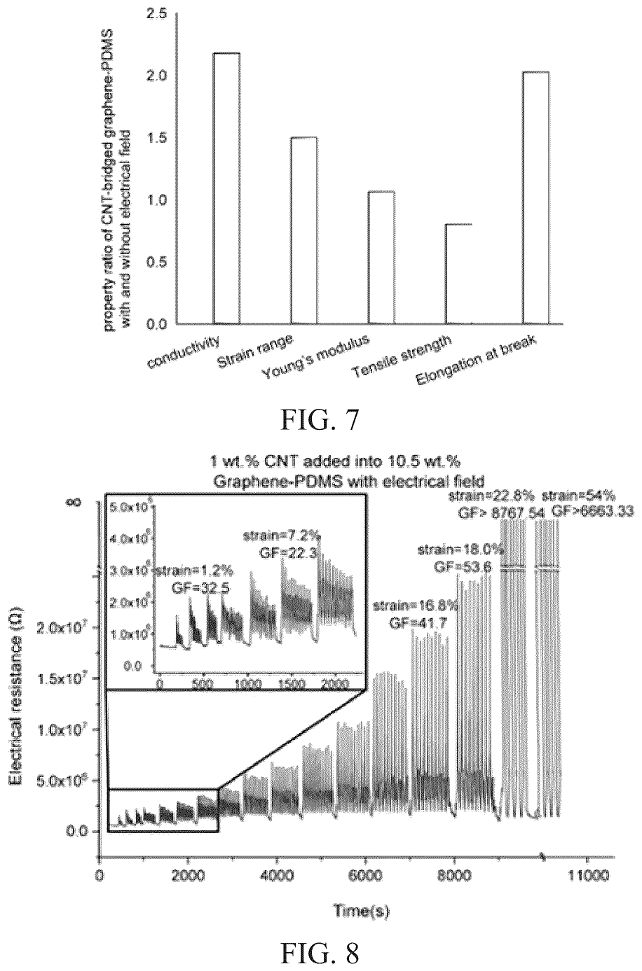

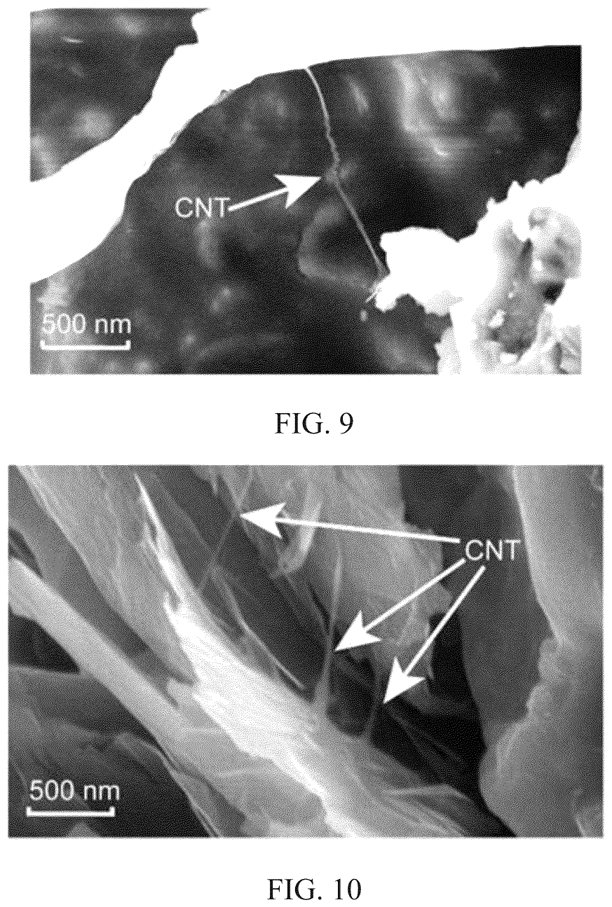

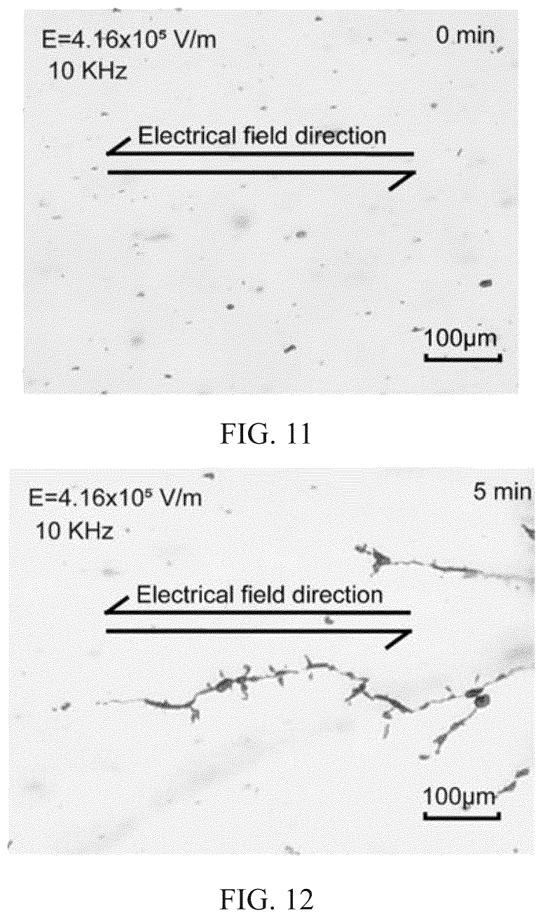

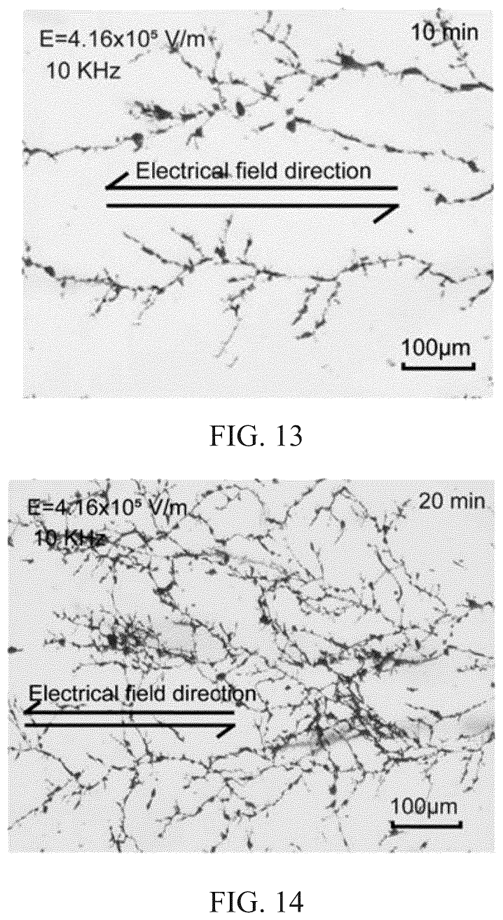

FIG. 7 to FIG. 14 explore effects of an electric field (4.16×10⁵ V/m, 10 kHz) on CNT-bridged polymeric material such as graphene-PDMS or PMMA and a mechanism thereof FIG. 7 is a property comparison (electrical conductivity, piezoresistive property and mechanical property) of CNT-bridged polymeric material such as graphene-PDMS or PMMA with or without an electric field. FIG. 8 is an example showing a peizoresistance test of aligned 1 wt % CNT-bridged polymeric material such as graphene-PDMS or PMMA induced by an electric field. FIG. 9 is scanning electron microscope (SEM) imaging showing that CNT bridges adjacent polymeric material clusters such as graphene-PDMS or PMMA. FIG. 10 is SEM imaging showing that aligned and parallel carbon nanotubes bridge adjacent polymeric material clusters such as graphene-PDMS or PMMA in an electric field direction. In a dynamic rearrangement process of CNT and graphene using a microscale electric field under an optical microscope (magnified by 100 times). FIG. 11 is 0 minute, FIG. 12 is 5 minutes, FIG. 13 is 10 minutes and FIG. 14 is 20 minutes after electric field treatment.

Random mixing of the CNT-Graphene-PDMS or PMMA and other polymeric materials will result in electron transfer paths in a one-dimensional to two-dimensional mixing network that are unconnected or connected in a single-end or overlapping mode in all directions. For this reason, an alternating electric field is applied to induce the arrangement of CNT-graphene in a curing process of the polymeric material such as PDMS or PMMA. A high-frequency electric field is used for optimizing the CNT-Graphene-PDMS or PMMA and other polymeric materials.

By applying the electric field, the electrical conductivity and piezoresistive strain range are increased by 2.22 times (0.12 S/m vs 0.054 S/m) and 1.50 times (0-54% vs 0-30%) respectively without electric field alignment, as shown in FIG. 7. After applying the electric field. 1 wt % CNT-bridged polymeric material such as graphene-PDMS or PMMA may generate regular electrical resistance changes in a strain range of 0% to 36%. When the strain further increases from 36% to 54%, the electrical resistance reaches a maximum electrical resistance range of a detection instrument. When the strain is released, the resistance may be restored to an initial value, Young's modulus and elongation at break also show an improvement trend (Young's modulus is increased by 1.06 times, and elongation at break is increased by 2.03 times). The tensile strength is decreased by 24.63%.

In order to verify the arrangement of nanomaterials (CNT-graphene) in a polymer, CNT-bridged polymeric material samples such as graphene-PDMS or PMMA arranged are cut in an electric field direction. Cross-sectional microstructures of the samples are observed by using a field emission scanning electron microscope (HitachiRegulus8220, Tokyo, Japan). SEM imaging (FIG. 20) proves this arrangement trend in the electric field direction. In order to further observe this dynamic rearrangement process at a microscale under an optical microscope (magnification of 100 times), 1 wt % CNT and 10.5 wt % graphene are randomly dispersed into oil in a random initial direction. After applying the alternating electric field, the carbon nanotubes and graphene in a microscope view are forced to align in the electric field direction.

In general, by introducing the bridging effect and electric field enhancement of the carbon nanotubes at a permeation threshold of the polymeric material such as graphene-PDMS or PMMA, it is compared with a traditional dry-blended method. In addition, the electrically aligned carbon nanotube bridged polymer material such as graphene-PDMS or PMMA is also superior to polymer materials such as graphene-PDMS or PMMA prepared by a solvent-based method in terms of strain coefficient and linear strain range.

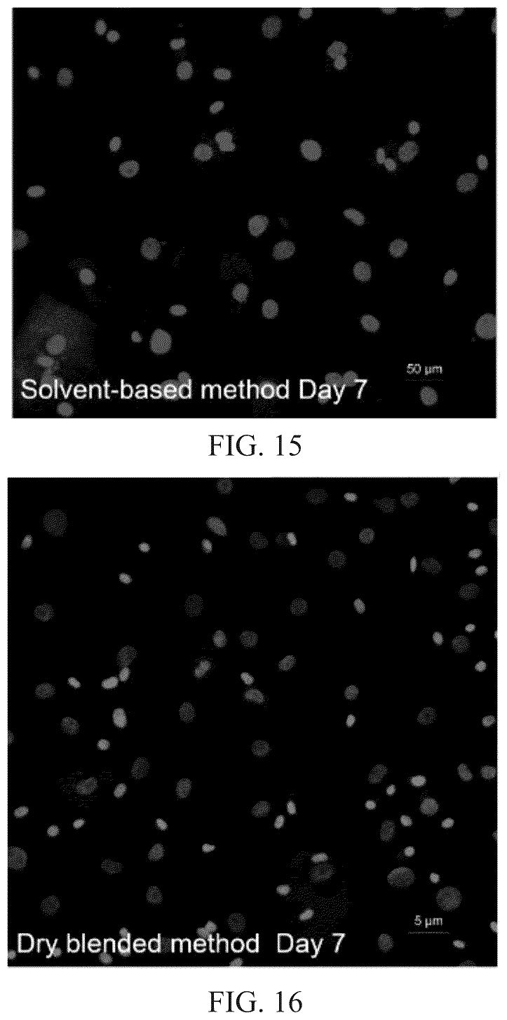

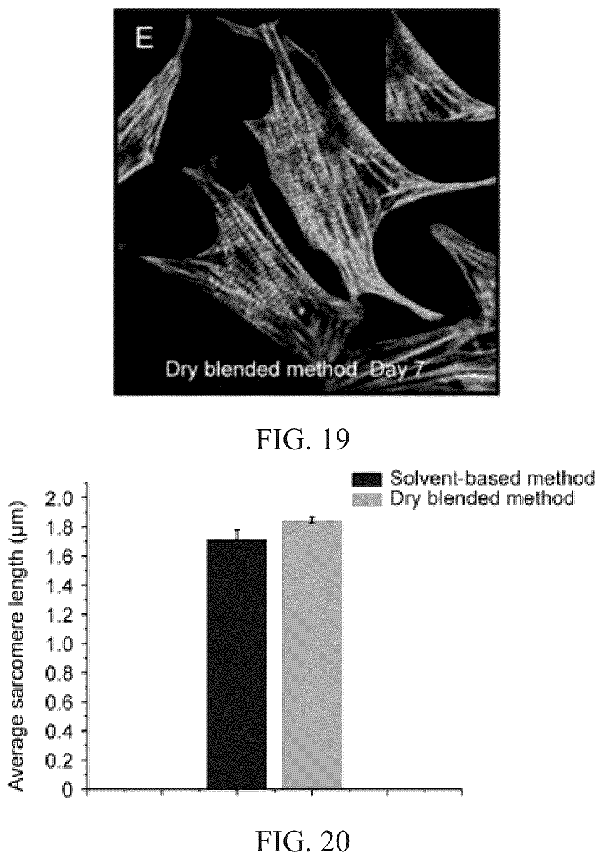

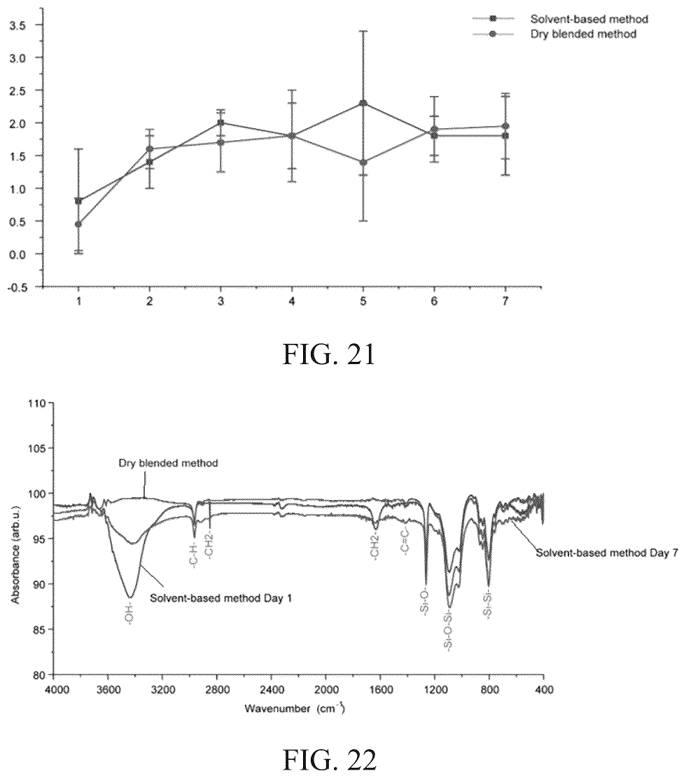

The biocompatibility of cardiomyocytes cultured on carbon nanotube bridged polymeric material such as graphene PDMS or PMMA and the solvent-based polymeric material such as graphene PDMS or PMMA, FIG. 15 and FIG, 16 are stained nuclei of 7-day cardiomyocytes: on CNT-bridged polymeric material such as graphene PDMS or PMMA and a polymeric material such as graphene PDMS or PMMA prepared by an organic solvent-based method, a scale is 50 μm. FIG. 17 is comparison of a density of cells cultured on CNT-bridged polymeric material such as graphene PDMS or PMMA and the polymeric materials such as graphene PDMS or PMMA by solvent-based method on the first and seventh days. FIG. 18 and FIG. 19 are carbon nanotube bridged polymeric material such as graphene PDMS or PMMA and the solvent-based polymeric material such as graphene PDMS or PMMA in stained α-actinin confocal images of cardiomyocytes after 7 days of culture, and a scale is 20 μm. FIG. 20 is an average sarcomere length, measured on the seventh day, of cardiomyocytes cultured on CNT-bridged polymeric material such as graphene PDMS or PMMA and the solvent-based polymeric material such as graphene PDMS or PMMA (1.66±0.11 μm and 1.86±0.10 μm). FIG. 21 is cardiomyocytes cultured on CNT-bridged polymeric material such as graphene PDMS or PMMA and the solvent-based polymeric material such as graphene PDMS or PMMA from the first day to the seventh day. During a culture period from the first day to the seventh day, (H) CNT bridges the polymeric material such as graphene PDMS or PMMA and a solvent FT-IR spectrum is based on the polymeric material such as graphene PDMS or PMMA (the first day and the seventh day).

The electrically aligned carbon nanotube bridged polymeric material such as graphene-PDMS or PMMA will be manufactured into a flexible sensor for long-term measurement of biological signals on the surface of organs or cells. Therefore, the potential biocompatibility of the polymeric material such as graphene-PDMS or PMMA is of high interest. in this embodiment, cardiomyocytes of a neonatal rat are cultured (for a total of 7 days) on surfaces of the polymeric composite materials such as graphene-PDMS or PMMA respectively. An experimental group adopts the proposed method for preparation, a control group adopts a traditional solvent-based method for preparation (n-hexane is a solvent, SD rat half lethal dose=15.8 g/kg), and curing evaporation time is 5 hours at 85° C. The cell density (a ratio of the nuclear number to a culture area), a survival rate (a ratio of the cell density on the seventh day and the first day) and an average sarcomere length of the cardiomyocytes of the neonatal rat and an average beating frequency of the cardiomyocytes of the neonatal rat are measured by the optical microscope, namely, a microscope (Olympus, CKX53) after immunofluorescent staining (nuclear and α-actinin) and a confocal microscope (Nikon, eclipseTI2).

After 24 hours, the cell density of the experimental group is 1.11 times that of the control group (0.42×10⁵±0.82×10⁴/cm² vs 0.38×10⁵±0.46×10⁴/cm²). After 7 days, the cell density of the experimental group is 1.64 times that of the control group (0.41×10⁵±1.04×10⁴/cm² vs 0.25×1.0⁵±0.75×10⁴/cm²), The 7-day cell survival rate of the experimental group is 1.47 times that of the control group (97.17% vs 65.96%).

The sarcomere is a basic unit of a contractile force of the cardiomyocytes, which helps to complete the regular contraction of the heart. α-actinin is a sarcomere component that can be observed by staining. A length of α-actinin is calculated according to a confocal microscope image. After 7 days of culture, the average sarcomere length of the cardiomyocytes of the experimental group is 1.86±0.51 !μm. In contrast, the average sarcomere length of the cardiomyocytes of the control group is 1.66±0.96 μm. In the culture process from the first day to the seventh day, the beating frequency of the cardiomyocytes of the experimental group is increased from 0.45±0.37 Hz to 1.95±0.49 Hz. Correspondingly, the cell value cultured by the control group is increased from 0.81±0.63 Hz to 1.82±0.58 Hz.

A Fourier transform infrared spectroscopy (Bruker, Nano-FTIR) is adopted to detect toxic residues of n-hexane. Common vibration signals of a CH bond, a Si—O bond, a C—Si bond and a CC bond at the wavelengths of 2954 cm⁻¹, 1257 cm⁻¹, 1088 cm⁻¹ and 796 cm⁻¹ respectively observe 1400 cm⁻¹ in all experimental group samples, which is related to the existence of the polymeric material such as PDMS or PMMA, graphene and CNT. Special vibration peak signals are observed at the wavelengths of 3450 cm⁻¹, 2850 cm⁻¹ and 1640 cm⁻¹ by the composite materials in the control group prepared by the solvent-based method. on the first day and the seventh day. This indicates that a —CH2— bond of n-hexane is used for vibration in a dispersion process of graphene, which indicates that the toxic residual hexane always remains in the polymeric material such as graphene PDMS or PMMA prepared by the solvent-based method. However, these vibration peaks are not observed in electrically arranged CNT-bridged polymeric material sample such as graphene-PDMS or PMMA. Due to avoiding the use of n-hexane, these results prove that the electrically aligned CNT-bridged polymeric material such as graphene-PDMS or PMMA has good biocompatibility.

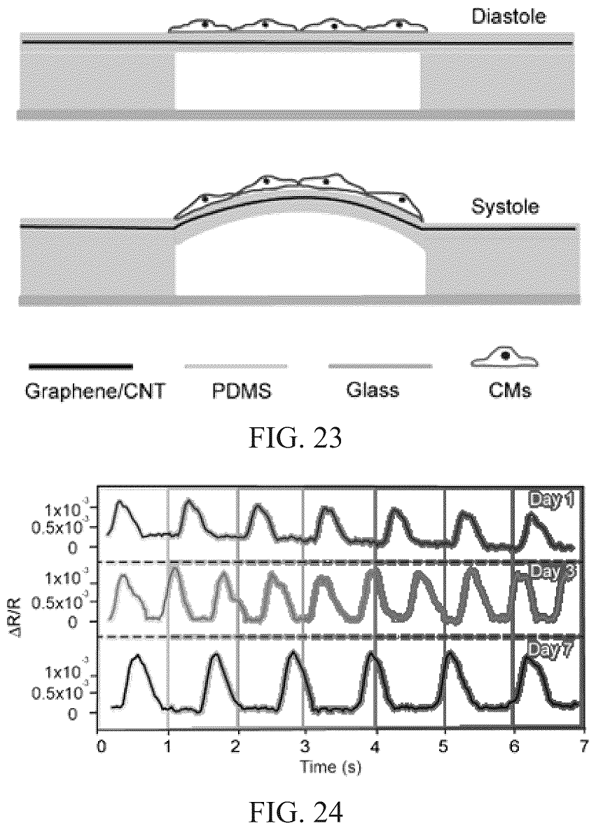

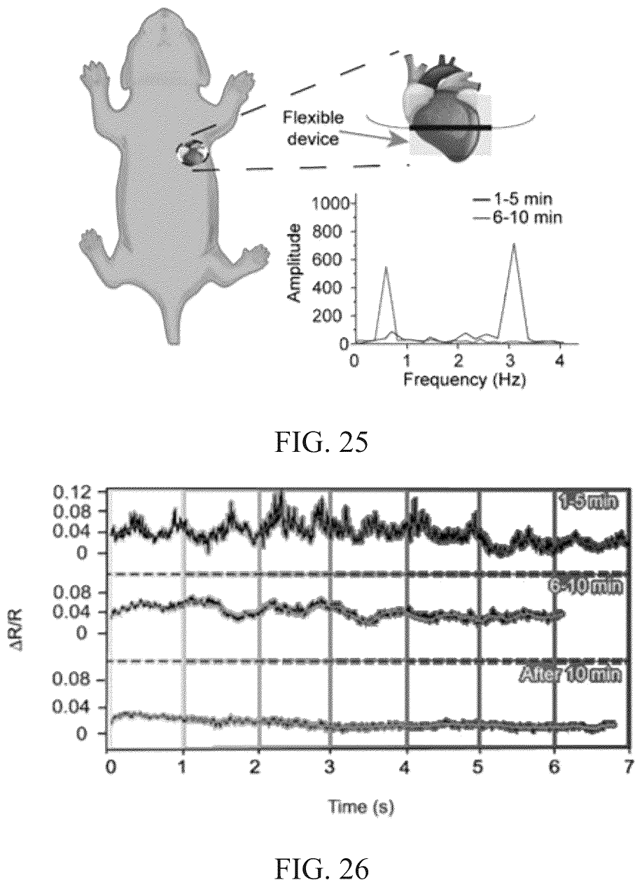

Application of carbon nanotube bridged polymeric material such as graphene PDMS or PMMA in the measurement of mouse myocardial and mouse cardiac contractions. A schematic diagram of FIG. 23 illustrates membrane deflection caused by diastolic and systolic phases of cardiac contraction at a cellular level. FIG. 24 is an example of measurement of ΔR/R0 caused by cardiomyocytes contraction in a culture process, and a CNT electrical resistance signal on the first, third and fifth days. FIG. 25 shows a schematic diagram (organ level) of measurement of cardiac contraction in a mouse heart and connecting a flexible device to a heart surface of an anesthetized rat. FIG. 26 is measurement of ΔR/R0 generated by cardiac contraction until the anesthetized rat stops beating. Exemplary CNT electrical resistance signals at the first 5 minutes, 5 to 10 minutes and 10 minutes after thoracotomy. The illustration shows Fourier transform of cardiac contraction signals, with the beating frequency decreasing from 3.07±0.3 Hz to 0.61±0.44 Hz.

The composite material proposed in this patent has advantages such as excellent flexibility, high sensitivity, tensibility and biocompatibility. It is very suitable for monitoring mechanical and electrical physiological signals. To this end, cellular-level and organ-level cardiac contraction measurements are used to evaluate properties of a novel flexible sensor of the polymeric materials such as CNT-Graphene-PDMS or PMMA.

The flexible sensor is manufactured by a proposed novel manufacturing method for measuring cardiac contractility in vitro (cellular level) and in vivo (organ level). This device is composed of a polymeric material film such as PDMS or PMMA with a thickness of 20 μm and an embedded strain dependent strip of CNT-bridged polymeric material such as graphene PDMS or PMMA. When the cardiomyocytes are cultured on the device film (a schematic diagram in FIG. 23), cells apply compressive stress on a top surface of the polymeric material film such as PDMS or PMMA during synchronous contraction. The stress causes membrane deflection and changes in the electrical resistance of CNT-bridged polymeric material such as graphene PDMS or PMMA. Sensor signals and electrical resistance changes (ΔR/R0) caused by cell beating increase from 3.43×10⁻⁵±1.71×10⁻⁶ of the first day to 1.84×10⁻⁴±3.25×10⁻⁵ of the fifth day. Correspondingly, the beating frequency of the cardiomyocytes is consistent with the beating frequency of cells in the 2.4^{th} sarcomere. The contraction force generated by the cardiomyocytes of the neonatal rat is far lower than that of common physiological activities in humans (for example, a pressure generated by single-layer rat cardiomyocytes is about <0.4 kPa, while a human wrist pulse force is about 6 kPa), which indicates that the flexible sensor is manufactured by using the proposed new manufacturing method and has excellent properties in terms of sensitivity and detection limits.

The sensor is attached to the heart surface of the anesthetized rat. Each cardiac contraction may generate a resistance change in the sensor. In the first 5 minutes after thoracotomy, an average pulsation frequency of the mouse is 3.07±0.3 Hz, and an average electrical resistance change (ΔR/R0) is 0.025±0.003, From 5 minutes to 10 minutes, the heartbeat frequency is decreased to 0.61±0.44 Hz, and the average electrical resistance change is 0.031±0.007. In 10 minutes, the heart of the mouse stops beating according to signals. The results indicate that the flexible sensor may be used for monitoring cardiac contraction in the rat. The above application also proves the biocompatibility of the proposed. green manufacturing method.

### Embodiment 2

this embodiment provides a flexible sensor, prepared by the method described in embodiment 1.

The above is only specific embodiments of the present disclosure, and is not intended to limit the present disclosure, and modifications and variations can be made in the present disclosure for those skilled in the art. Any modification, equivalent substitution, improvement, and the like made within the spirit and principle of the present disclosure shall be encompassed within the protection scope of the present disclosure.

1. A method for manufacturing a flexible sensor, comprising:
dispersing graphene in a polymeric material;
adding a carbon nanotube into the polymeric material;
applying an alternating electric field to the polymeric material added with the carbon nanotube to obtain a composite material;
attaching a polymeric material film to the obtained composite material;
pre-embedding carbon fibers; and
heating and curing to obtain a sensor.

2. The method for manufacturing the flexible sensor according to claim 1, wherein the graphene is dispersed in the polymeric material in a mode of mechanical stirring and high-power ultrasound by using a dry-blended method.

3. The method for manufacturing the flexible sensor according to claim 1, wherein a mechanical stirring duration is 0.5 hour to 24 hours.

4. The method for manufacturing the flexible sensor according to claim 1, wherein the polymeric material is polydimethylsiloxane or polymethyl methacrylate.

5. The method for manufacturing the flexible sensor according to claim 1, wherein a mass ratio of the graphene is 2 wt % to 30 wt %; and a mass ratio of the carbon nanotube is 0.1 wt % to 10 wt %.

6. The method for manufacturing the flexible sensor according to claim 1, wherein the alternating electric field is set as a sinusoidal complex-frequency alternating electric field with an intensity of 10⁴ to 10⁶ V/m and a frequency of 100 Hz to 10 KHz.

7. The method for manufacturing the flexible sensor according to claim 1, wherein a curing agent is added into the mixed composite material before use, and a mass ratio of the composite material to the curing agent ranges from 5 wt % to 20 wt %.

8. The method for manufacturing the flexible sensor according to claim 1, wherein a process of attaching the polymeric material film to the obtained polymeric material is that the composite material is put into a mold, and the polymeric material film is attached to a top of the mold.

9. The method for manufacturing the flexible sensor according to claim 1, wherein a process of heating and curing is that the polymeric material film is compacted, the carbon fibers are embedded at two ends, and curing is performed at 70° C. for 5 hours.

10. A flexible sensor, prepared by the method according to claim 1.
